(19) United States
(12) Patent Application Publication
(10) Pub. No.: US 2019/0201498 A1
(43) Pub. Date: Jul. 4, 2019

(54) USE OF EARTHWORM PROTEIN FOR PREPARING PHARMACEUTICAL COMPOSITION FOR PROTECTION OF BRAIN NEURONS

(71) Applicant: TCI Co., Ltd., Taipei (TW)

(72) Inventors: Yung-Hsiang LIN, Taipei (TW); Yu-Hung SU, Taipei (TW)

(21) Appl. No.: 16/205,731

(22) Filed: Nov. 30, 2018

(30) Foreign Application Priority Data
Dec. 29, 2017 (TW) 106146634

(51) Int. Cl. A61K 38/44; A61P 25/28; A61K 38/17

(52) U.S. Cl. CPC A61K 38/446; C12Y 115/01001; A61K 38/1767; A61P 25/28

(57) ABSTRACT

The present invention relates to a use of earthworm protein. Particularly, the present invention provides a use of earthworm protein for preparing a pharmaceutical composition which can enhance the expression of an antioxidant gene in brain neurons or inhibit the effects of β-amyloid protein (Aβ) and 1-methyl-4-phenylpyridinium (MPP⁺) on brain neurons. As such, the pharmaceutical composition comprising the earthworm protein has effects of reducing damage to brain neurons.

### RELATED APPLICATIONS

This application claims priority to Taiwanese Patent Application Number 106146634, filed on Dec. 29, 2017, which is incorporated herein by reference.

### TECHNICAL FIELD

The present invention relates to a use of earthworm protein, and more particularly, to a use of earthworm protein for preparing a pharmaceutical composition for protection of brain neurons.

### BACKGROUND ART

According to an estimate made by the Alzheimer's Disease International (ADI) in 2017, ten million new cases of dementia would be reported in 2017, equivalent to one person being diagnosed of dementia every 3 seconds on average for the year. In 2017, the global population of people who are suffering from dementia is approximately as high as fifty million, and the population is estimated to reach 131.5 million in 2050 Alzheimer's disease is the most common dementia and has early symptoms such as lapse of memory, having difficulties in the recognition of time, places and people and exhibiting more than two types of cognitive impairments, which are progressive and irreversible degeneration. In addition, the brain neurons of a patient would be damaged: hippocampus is the main afflicted region in the early stage, and abnormal senile plaque and neurofibrillary tangle occur in later stages, seriously affecting the autonomy and self-awareness abilities of the patient, and also severely impacting patient care at home. Therefore, it would be a great benefit for patients suffering from Alzheimer's disease if a pharmaceutical composition having a preventive effect or even a remedial effect against Alzheimer's disease can be found.

Similarly, Parkinson's disease is also a serious disease related to dementia. In 2015, there were approximately 6.2 million patients suffering from Parkinson's disease globally, in which 117,000 people died consequently. According to statistics, approximately 1% of elderly people above the age of 60 would be afflicted by the disease. The disease is a chronic degenerative disease of the central nervous system and mainly affects the motor nervous system. Symptoms of the disease slowly appear with lapse of time, and the most obvious symptoms in the early stage include tremor, rigidity of limbs, hypokinesia and abnormal gaits, and dementia is most commonly observed in severely afflicted patients. The motor symptoms of Parkinson's disease are mainly a result of death of substantia nigra cells, which leads to insufficient dopamine in the brain region of a patient. Since Parkinson's disease cannot be cured at the moment, the motor symptoms can only be controlled by medicines or reduced by surgeries. Therefore, a discovery of a pharmaceutical composition having a preventive or even remedial effect against Parkinson's disease is urgently needed for reducing the number of patients suffering from Parkinson's disease or mitigating their symptoms, or even lowering their death rate.

Earthworms (genus Pheretima) are traditionally used as a traditional Chinese medicinal material, and are generally considered to possess effects such as promoting blood circulation to remove blood stasis and preventing and treating cardiovascular diseases. Earthworm protein is an extract from earthworms. Apart from containing trace elements such as iron, manganese, copper, zinc and selenium, the earthworm extract is also rich in many peptides and enzymes. It is currently known that the collagenase, plasmin and lumbrokinase contained in earthworm protein have certain effects on dissolving blood clots, and thus can be applied for preventing cardiovascular diseases or improving sequela of apoplexy. The specific effects of other components in earthworm protein are still being researched.

### SUMMARY

An objective of the present invention is to provide a use of earthworm protein as a component in a pharmaceutical composition for protection of brain neurons. By using embodiments provided by the present invention, earthworm protein can be used to prepare a pharmaceutical composition for reducing damage to brain neurons.

In order to achieve the above objective, the present invention provides a use of earthworm protein for preparing a pharmaceutical composition for protection of brain neurons, wherein the earthworm protein does not include lumbrokinase, but the pharmaceutical composition for protection of brain neurons prepared by using the earthworm protein can be used to reduce damage to brain neurons by enhancing the expression of antioxidant genes in neurons, or by inhibiting effects of β-amyloid protein (Aβ) and 1-methyl-4-phenylpyridinium (MPP⁺) on neurons.

In an embodiment of the present invention, the pharmaceutical composition for protection of brain neurons can be used to improve antioxidative capability of neurons.

In an embodiment of the present invention, the pharmaceutical composition for protection of brain neurons can be used to enhance the expression of an antioxidant gene in neurons.

In an aspect of an embodiment of the present invention, the antioxidant gene may be superoxide dismutase 1 (SOD1), superoxide dismutase 2 (SOD2) or glutathione peroxidase (GPX).

In an embodiment of the present invention, the concentration of the earthworm protein comprised in the pharmaceutical composition for enhancing the expression of the antioxidant gene in neurons may be approximately 0.05-0.7 mg/ml.

In another embodiment of the present invention, the pharmaceutical composition for protection of brain neurons can be used to inhibit damage of β-amyloid protein to brain neurons. In one aspect, the pharmaceutical composition for protection of brain neurons can be used to prevent Alzheimer's disease, wherein the concentration of the earthworm protein comprised in the pharmaceutical composition may be approximately 0.005-0.05 mg/ml.

In another embodiment of the present invention, the pharmaceutical composition for protection of brain neurons can be used to inhibit damage of 1-methyl-4-phenylpyridinium to brain neurons. In one aspect, the pharmaceutical composition for protection of brain neurons can be used to prevent Parkinson's disease, wherein the concentration of the earthworm protein comprised in the pharmaceutical composition may be approximately 0.04-0.08 mg/ml.

In an embodiment of the present invention, the earthworm protein comprised in the pharmaceutical composition for protection of brain neurons is obtained by extraction from an earthworm with water. In one aspect, the earthworm protein comprised therein is obtained by extraction from the earthworm with warm water of approximately 40-50° C.

In another embodiment of the present invention, the earthworm may be Pheretima aspergillum (E. Perrier), Pheretima vulgaris Clen, Eisenia rosea savigny, Eisenia Foetida, Pheretima pectinifera or Pherertima guillelmi.

The embodiments of the present invention will be further explained below. The following enumerated embodiments are used for the purpose of elucidating the present invention, rather than to limit the scope of the present invention. One skilled in the art may make several changes and modifications without departing from the spirit and scope of the present invention. Therefore, the protection scope of the present invention shall subject to the appended claims.

### BRIEF DESCRIPTIONS OF THE DRAWINGS

FIG. 1 is a diagram showing a result of an antioxidation experiment for an earthworm protein extract according to an embodiment of the present invention.

FIG. 2 is a diagram showing a result of the effect of an earthworm protein extract according to an embodiment of the present invention on the expression of SOD1 gene.

FIG. 3 is a diagram showing a result of the effect of an earthworm protein extract according to an embodiment of the present invention on the expression of SOD2 gene.

FIG. 4 is a diagram showing a result of the effect of an earthworm protein extract according to an embodiment of the present invention on the expression of GPX gene.

FIG. 5 is a diagram showing a result of inhibition of an earthworm protein extract according to an embodiment of the present invention on damage of Aβ to neurons.

FIG. 6 is a diagram showing a result of inhibition of an earthworm extract according to an embodiment of the present invention on damage of MPP⁺ to neurons.

### DETAILED DESCRIPTIONS

Earthworm protein is an extract extracted by processing earthworms, and may be obtained by using the following method. Since extraction methods of earthworm protein are already well known to a person of ordinary skill in the art, the following extraction method is merely an example and the present invention is by no means limited thereto. Optimal concentrations are as shown below.

### Embodiment 1: Preparation of Earthworm Protein

Earthworms were put into water and stirred in order to wash silt, impurities or mucus off the earthworms, and washing was continued until water from the washing became clear. Before washing, an emetic treatment may be performed on the earthworms, so as to allow the earthworms to expel undigested food or impurities inside their bodies. Afterwards, the washed and cleaned earthworms were put into an ozone solution for sterilization, taken out for washing again and then immersed in warm water of 40-50° C., wherein a ratio of the earthworms to water was 1:20-1:5, preferably 1:12-1:8. After leaving the earthworms in the warm water at 40-50° C. for 12-16 hours, the earthworms were grinded using a grinder. A pulp solution resulted from the grinding was firstly filtered using a 50-100 mesh screen cloth. After a filtered solution was collected, the solution was centrifuged for 8-28 minutes at 6000-14000 rpm (which were adjustable according to the volume of the solution), followed by obtaining and filtering a supernatant thereof and then performing another round of centrifugation as described above, and a supernatant was obtained, giving an earthworm extract, which is the earthworm protein referred to herein. Upon testing, it is found that the earthworm protein does not include lumbrokinase. The solution of the earthworm extract described previously may further be concentrated using an ultrafiltration membrane, or further lyophilized to form a powder to be used for subsequent experiments or uses.

The aforementioned earthworms may be animals belonging to the class of Oligochaeta in the phylum Annelida, such as Pheretima aspergillum (E. Perrier), Pheretima vulgaris Clen, Eisenia rosea savigny, Eisenia Foetida, Pheretima pectinifera or Pherertima guillelmi, but is not limited thereto.

### Embodiment 2: Antioxidation Effect of Earthworm Protein on Brain Neurons

Firstly, the earthworm protein prepared in Embodiment 1 was formulated with pure water into a solution having a concentration of 0.05-0.7 mg/ml, preferably 0.5 mg/ml.

A mouse brain neuroma cell strain (Neuro-2a, ATCC, CCL-131) was prepared and cultured in a cell culture solution [Dulbecco's modified Eagle's medium (DMEM), 1% Penicillin-streptomycin (Gibco), and 10% fetal bovine serum (Gibco)]. A 6-well cell culture plate was prepared, and 2 ml of the cell culture solution was planted into each well, such that each well had 1.5×10⁵ cells, and the cells were cultured for 24 hours at 37° C.

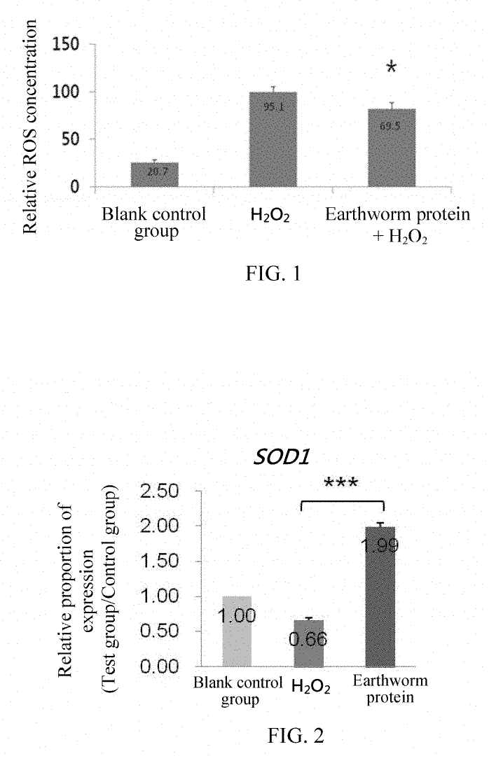

Subsequently, without affecting the adhered cells, the culture solution in each well was removed, and the wells were divided into three groups (2 wells for each group) for experiments, in which Group A was a blank control group, Group B was added with 500 μM of H₂O₂ (Sigma), and Group C was added with 500 μM of H₂O₂ and 0.5 mg/ml of the earthworm protein extract prepared in Embodiment 1 above. After the above groups were added into the wells together with culture solutions, the cells were cultured for 1 hour at 37° C. Afterwards, 5 μg/ml of DCFH-DA (Sigma/SI-D6883-50MG) was further added into each well for reaction for 15 minutes at 37° C. Subsequently, H₂O₂ was allowed to react for 1 hour at 37° C. The cells in each well were then rinsed twice using 1 ml of 1× PBS (Gibco). After rinsing, 200 μl of trypsin was separately added into each well for reaction in the dark for 5 minutes. The cell solutions were then removed and placed into 1.5 ml centrifugation tubes and centrifuged for 10 minutes at 400×g. The supernatant was removed, and the tubes were rinsed once with 1× PBS and centrifuged again for 10 minutes at 400×g. The supernatant was removed again, and cell precipitants were suspended in 1 ml of 1× PBS. Afterwards, a flow cytometry was used to detect fluorescent signals of DCFH-DA at an excitation wavelength (450-490 nm) and an emission wavelength (510-550 nm) respectively. The detected values were input into Microsoft Excel software and statistical significance between the two values was analyzed using Student's t-test, and a result thereof is shown in FIG. 1. In comparison to H₂O₂, the p-value of the group indicated by *is <0.05.

It can be concluded from the result shown in FIG. 1 that under the condition in which the earthworm protein extract of the present invention is included, the concentration of reactive oxygen species is significantly reduced by approximately 25%, which confirms that the earthworm protein extract of the present invention is capable of decreasing generation of free radicals like reactive oxygen species, thereby reducing damage of the reactive oxygen species to brain neurons.

### Embodiment 3: Effects of Earthworm Protein on Expression of Antioxidant Genes in Brain Neurons

Firstly, the earthworm protein prepared in Embodiment 1 was formulated with a normal saline solution into a solution having a concentration of 0.05-0.7 mg/ml, preferably 0.5 mg/ml. Human skin fibroblast cells (CCD-966sk, BCRC No. 60153) were also prepared, and 2 ml of the cell culture solution was planted into each well of a 6-well plate, such that each well had 1.5×10⁵ cells, and the cells were then cultured for 24 hours at 37° C. Subsequently, without affecting the adhered cells, the culture solution in each well was removed, and the wells were divided into the following groups for experiments, which respectively includes: a blank control group, a group having only H₂O₂ added thereto (H₂O₂ only), and a group having H₂O₂ and 0.5 mg/ml of the earthworm protein extract prepared in Embodiment 1 added thereto (earthworm protein). The cells were cultured for 24 hours at 37° C. before analysis of the expression of specific genes.

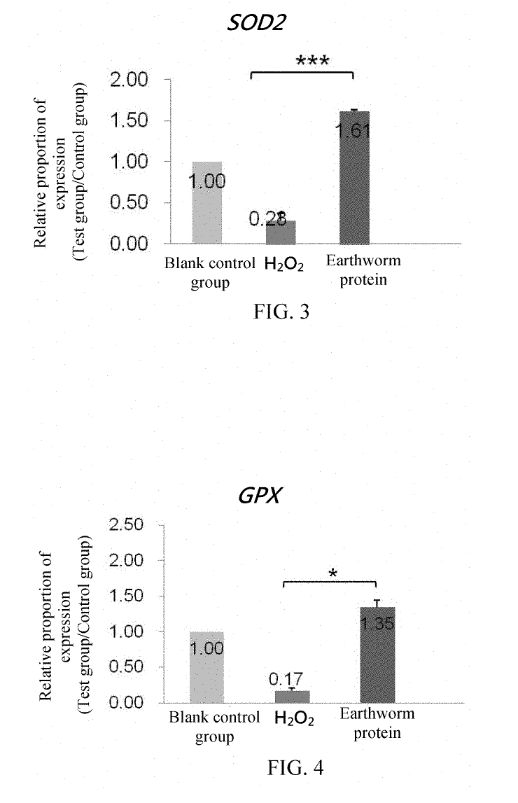

The foregoing groups of cells were recycled, and RNAs were extracted therefrom using an RNA extraction kit (Geneaid), and the RNAs were reversely transcribed into cDNAs using a reverse transcriptase (Invitrogen). Afterwards, by using a Real-Time PCR system (ABI Step One Plus Real-Time PCR system), qPCR (KAPA CYBR FAST qPCR Kits, KAPA Biosystems) was performed using the primers listed in Table 1 respectively, so as to quantify the expression of the following genes: SOD1, SOD2 and GPX. Relative quantitative analyses on gene expression were conducted using a 2-^{ΔΔCt} method. The results related to the expression of the genes are shown in FIGS. 2 to 4, wherein the p-values of the groups indicated by * are <0.05, and the p-values of the groups indicated by *** are <0.001.

TABLE 1

| | | Sequence | Primer Length | Product Length |
|---|---|---|---|---|
| Gene | Primer | Number | (ntds) | (ntds) |
| SOD1 | SOD1-F | SEQ ID NO: 1 | 21 | 227 |
| | SOD1-R | SEQ ID NO: 2 | 20 | |
| SOD2 | SOD2-F | SEQ ID NO: 3 | 21 | 116 |
| | SOD2-R | SEQ ID NO: 4 | 23 | |
| GPX | GPX1-F | SEQ ID NO: 5 | 21 | 105 |
| | GPX1-R | SEQ ID NO: 6 | 19 | |

It can be concluded from the results shown in FIGS. 2 to 4 that, under the condition in which the earthworm protein extract of the present invention is included and left to take effect for 24 hours, a relative value of SOD1 gene expression is increased from 0.66 to 1.99, a relative value of SOD2 gene expression is increased from 0.28 to 1.61, and a relative value of GPX gene expression is increased from 0.17 to 1.35. Therefore, the earthworm protein extract of the embodiment of the present invention can indeed induce increases in expressions of antioxidant genes such as SOD1, SOD2 and GPX.

### Embodiment 4: Inhibitory Effect of Earthworm Protein on Damage of Aβ to Neurons

The earthworm protein prepared in Embodiment 1 was formulated with a normal saline solution into a solution having a concentration of 0.005-0.05 mg/ml, preferably 0.008 mg/ml. A human neuroblastoma cell strain (SH-SY5Y, ATCC CRL-2266) was also prepared, and 2 ml of the cell culture solution was planted into each well of a 6-well plate, such that each well had 2×10⁴ cells, and the wells were divided into the following groups for experiments, which respectively include: a blank control group, a β-amyloid peptide (Lifetein) group (Aβ only), a Methyllycaconitine (MLA) control group added with Aβ (PC+Aβ), and a group added with 0.008 mg/ml of the earthworm protein extract prepared in Embodiment 1 and Aβ (earthworm protein+Aβ). The cells were cultured for 24 hours at 37° C. before analysis of survival rates of brain neurons.

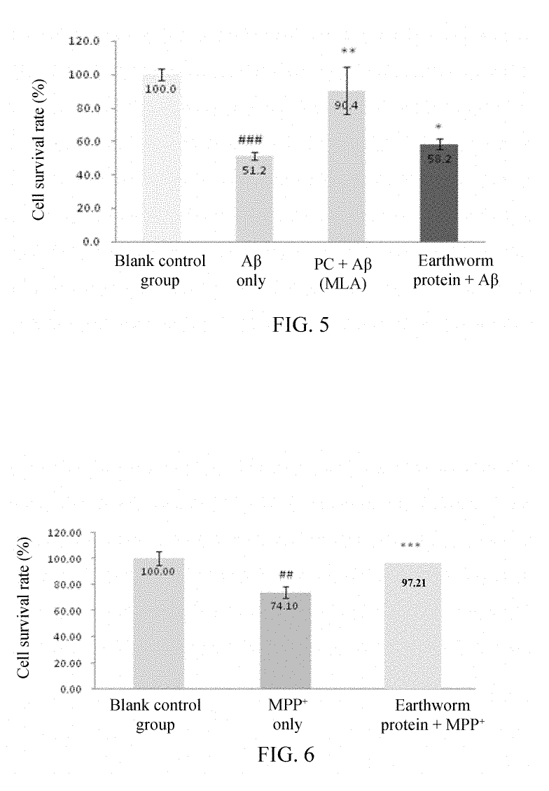

Firstly, 15 μl of an MTT (thiazoyl blue tetrazolium bromide, AMRESCO/0793-5G) solution was added into each well, and the cells were cultured for 4 hours at 37° C. The solution was subsequently removed and 50 μl of a DMSO (ECHO/DA1101-000000-72EC) solution was added into each well so as to dissolve formazan formed by reaction. The 6-well plate was placed on a shaker and shaken for 10 minutes, and then an ELISA analyzer (BioTek) was used to measure absorbance values at a wavelength of 570 nm, and cell survival rates (%) were calculated using the following formula: (an absorbance value of a test group/an absorbance value of a control group)×100%. Finally, the values were input into Microsoft Excel software and statistical significance between the values was analyzed using Student's t-test, and a result thereof is shown in FIG. 5. In comparison to the Aβ only group, the p-value of the group indicated by * is <0.05, the p-value of the group indicated by ** is <0.01; in comparison to the blank control group, the p-value of the group indicated by ### is <0.001.

It can be concluded from the result shown in FIG. 5 that, under the condition in which the earthworm protein extract of the embodiment of the present invention is included, the cell survival rate is increased from 51.2% to 58.2% and the amplitude of increase is approximately 7%, which proves that the earthworm protein extract of the embodiment of the present invention is capable of inhibiting the effects of Aβ on brain neurons and reducing the damage thereof; therefore, the earthworm protein extract can also be applied in the prevention of Alzheimer's disease.

### Embodiment 5: Inhibitory Effect of Earthworm protein on Damage of MPP to Neurons

The earthworm protein prepared in Embodiment 1 was formulated with a normal saline solution into a solution having a concentration of 0.04-0.08 mg/ml, preferably 0.06 mg/ml. A human neuroblastoma cell strain (SH-SY5Y, ATCC CRL-2266) was also prepared, and 2 ml of the cell culture solution was planted into each well of a 6-well plate, such that each well had 2×10⁴ cells, and the wells were divided into the following groups for experiments, which respectively include a blank control group, an MPP+ group (MPP⁺ only), and a group having 0.06 mg/ml of the earthworm protein extract prepared in Embodiment 1 and MPP added thereto (earthworm extract+MPP⁺). The cells were cultured for 24 hours at 37° C. before analysis of survival rates of brain neurons.

Firstly, 15 μl of an MTT (AMRESCO/0793-5G) solution was added into each well, and the cells were cultured for 4 hours at 37° C. The solution was subsequently removed and 50 μl of a DMSO (ECHO/DA1101-000000-72EC) solution was added into each well so as to dissolve formazan formed by reaction. The 6-well plate was placed on a shaker and shaken for 10 minutes, and then an ELISA analyzer (BioTek) was used to measure absorbance values at a wavelength of 570 nm, and cell survival rates (%) were calculated using the following formula: (an absorbance value of a test group/an absorbance value of a control group)×100%. Finally, the values were input into Microsoft Excel software and statistical significance between the values was analyzed using Student's t-test, and a result thereof is shown in FIG. 6. In comparison to the MPP only group, the p-value of the group indicated by *** is <0.001; in comparison to the blank control group, the p-value of the group indicated by ## is <0.01.

It can be concluded from the result shown in FIG. 6 that, under the condition in which the earthworm protein extract of the embodiment of the present invention is included, the cell survival rate is increased from 74.1% to 97.21%, a significant increase of approximately 23.11%, which proves that the earthworm protein extract of the embodiment of the present invention is capable of inhibiting the effects of MPP on brain neurons and reducing the damage thereof; therefore, the earthworm protein extract can also be applied in the prevention of Parkinson's disease.

The pharmaceutical composition for protection of brain neurons of the embodiments of the present invention can further be added with carriers or other adjuvants known in the art. The dosage form thereof may be, but is not limited to, solutions, capsules or tablets.

It can be proven from the above results that the earthworm protein extracts in the pharmaceutical compositions prepared in the embodiments of the present invention have excellent antioxidation effects for brain neurons, and can enhance the expression of antioxidant genes of the neurons and inhibit the effects of Aβ or 1-methyl-4-phenylpyridinium on brain neurons so as to reduce the damage to the brain neurons, and thus can further be used in pharmaceutical compositions for preventing Alzheimer's disease or Parkinson's disease.

What is claimed is:

1. A use of earthworm protein for preparing a pharmaceutical composition for protection of brain neurons, wherein the earthworm protein does not comprise lumbrokinase and is capable of reducing damage to brain neurons.

2. The use of claim 1, wherein the pharmaceutical composition for protection of brain neurons is used to enhance an expression of an antioxidant gene in neurons.

3. The use of claim 2, wherein the antioxidant gene is SOD1, SOD2 or GPX.

4. The use of claim 2, wherein the pharmaceutical composition for protection of brain neurons has a concentration of earthworm protein of approximately 0.05-0.7 mg/ml.

5. The use of claim 1, wherein the pharmaceutical composition for protection of brain neurons is used to inhibit damage of β-amyloid protein (Aβ) to neurons.

6. The use of claim 5, wherein the pharmaceutical composition for protection of brain neurons is used to prevent Alzheimer's disease.

7. The use of claim 5, wherein the pharmaceutical composition for protection of brain neurons has a concentration of earthworm protein of approximately 0.005-0.05 mg/ml.

8. The use of claim 1, wherein the pharmaceutical composition for protection of brain neurons inhibits damage of 1-methyl-4-phenylpyridinium (MPP⁺) to neurons.

9. The use of claim 8, wherein the pharmaceutical composition for protection of brain neurons is used to prevent Parkinson's disease.

10. The use of claim 8, wherein the pharmaceutical composition for protection of brain neurons has a concentration of earthworm protein of approximately 0.04-0.08 mg/ml.

11. The use of claim 1, wherein the earthworm protein comprised in the pharmaceutical composition for protection of brain neurons is obtained by extraction from an earthworm with water.

12. The use of claim 11, wherein the earthworm protein comprised in the pharmaceutical composition for protection of brain neurons is obtained by extraction from the earthworm with warm water of approximately 40-50° C.

13. The use of claim 11, wherein the earthworm is Pheretima aspergillum (E. Perrier), Pheretima vulgaris Clen, Eisenia rosea savigny, Eisenia Foetida, Pheretima pectinifera or Pheretima guillelmi.

14. The use of claim 2, wherein the pharmaceutical composition for protection of brain neurons has a concentration of earthworm protein of approximately 0.05-0.7 mg/ml.

15. The use of claim 6, wherein the pharmaceutical composition for protection of brain neurons has a concentration of earthworm protein of approximately 0.005-0.05 mg/ml.

16. The use of claim 9, wherein the pharmaceutical composition for protection of brain neurons has a concentration of earthworm protein of approximately 0.04-0.08 mg/ml.

17. The use of claim 12, wherein the earthworm is Pheretima aspergillum (E. Perrier), Pheretima vulgaris Clen, Eisenia rosea savigny, Eisenia Foetida, Pheretima pectinifera or Pheretima guillelmi.


SEQUENCE LISTING (6 sequences)

SEQ ID NO: 1 - DNA, 21 bp, Artificial sequence
  Primer
ggtgggccaaaggatgaagag

SEQ ID NO: 2 - DNA, 20 bp, Artificial sequence
  Primer
ccacaagccaaacgacttcc

SEQ ID NO: 3 - DNA, 21 bp, Artificial sequence
  Primer
ggaagccatcaaacgtgactt

SEQ ID NO: 4 - DNA, 23 bp, Artificial sequence
  Primer
cccgttccttattgaaaccaagc

SEQ ID NO: 5 - DNA, 21 bp, Artificial sequence
  Primer
cagtcggtgtatgccttctcg

SEQ ID NO: 6 - DNA, 19 bp, Artificial sequence
  Primer
gagggacgccacattctcg